(19) United States
(12) Patent Application Publication
(10) Pub. No.: US 2021/0015760 A1
(43) Pub. Date: Jan. 21, 2021

(54) PHARMACEUTICAL FORMULATIONS CONTAINING GABOXADOL FOR THERAPEUTIC TREATMENT

(71) Applicant: Ovid Therapeutics Inc., New York, NY (US)

(72) Inventor: Matthew DURING, Weston, CT (US)

(21) Appl. No.: 16/929,345

(22) Filed: Jul. 15, 2020

Related U.S. Application Data
(60) Provisional application No. 62/874,152, filed on Jul. 15, 2019.

(51) Int. Cl. A61K 9/70; A61K 9/20; A61K 9/48; A61K 9/00; A61K 31/437

(52) U.S. Cl. CPC A61K 9/7023; A61K 9/20; A61K 31/437; A61K 9/006; A61K 9/48

(57) ABSTRACT

Pharmaceutical formulations containing gaboxadol or a pharmaceutically acceptable salt thereof and methods of treating essential tremors, Tourette syndrome or Fragile X syndrome are provided. Pharmaceutical formulations herein include transdermal formulations and modified release dosage forms. In embodiments, a modified release dosage form includes an orally disintegrating dosage form. In embodiments, a modified release dosage form includes an extended release dosage form. In embodiments, a modified release dosage form includes a delayed release dosage form. In embodiments, a modified release dosage form includes a pulsatile release dosage form.

### CROSS-REFERENCE TO RELATED APPLICATION

This application claims benefit and priority to U.S. Provisional Application No. 62/374,152, filed Jul. 15, 2019, which is incorporated herein by reference in its entirety.

### TECHNICAL FIELD

Pharmaceutical formulations containing gaboxadol or a pharmaceutically acceptable salt thereof are provided.

### BACKGROUND

Gaboxadol (4,5,6,7-tetrahydroisoxazolo [5,4-c]pyridine-3-ol) (THIP)) is described in EP Patent No. 0000338 and in EP Patent No. 0840601, U.S. Pat. Nos. 4,278,676, 4,362,731, 4,353,910, and WO 2005/094820. Gaboxadol is a selective GABA_{A} receptor agonist with a preference for δ-subunit containing GABA_{A} receptors. Gaboxadol is an agonist of GABA receptors that contain α₄, α₆, and δ, subunits, which have more restricted anatomic distribution in the thalamus, hippocampus, and cerebellum and are mainly extrasynaptic in location. Gaboxadol has its greatest efficacy at α4βδ and α6βδ GABA_{A} receptors, that is, benzodiazepine-insensitive receptors that contribute to tonic inhibitory conductances rather than synaptic inhibitory postsynaptic currents. Accordingly, the mode of action and effects of gaboxadol are distinct from those of benzodiazepine receptor agonists. Extrasynaptic GABA receptors are sensitive to low concentrations of GABA, they desensitize slowly, and their activation can induce sustained neuronal effects. In conventional pharmaceutical formulations such as tablets and capsules, gaboxadol is rapidly absorbed, reaching peak concentration within 30 minutes, with a half-life of approximately 1.5 to 2 hours. Gaboxadol is a zwitterion with pKa values of 4.3 (acidic) and 8.3 (basic) and log P of 0.61. Gaboxadol is highly soluble, more than 30 mg/mL in the physiological pH range.

In the early 1980s gaboxadol was the subject of a series of pilot studies that tested its efficacy as an analgesic and anxiolytic, as well as a treatment for tardive dyskinesia, Huntington's disease, Alzheimer's disease, and spasticity. In the 1990s gaboxadol moved into late stage development for the treatment of insomnia. The development was discontinued after the compound failed to show significant effects in sleep onset and sleep maintenance in a three-month efficacy study. Additionally, patients with a history of drug abuse who received gaboxadol experienced a steep increase in psychiatric adverse events.

According to the National Institutes of Health, National Institute of Neurological Disorders and Stroke (https://www.ninds.nih.gov/Disorders/All-Disorders/Essential-Tremor-Information-Page), tremor is an unintentional, somewhat rhythmic, muscle movement involving to-and-fro movements (oscillations) of one or more parts of the body. Essential tremor (previously called benign essential tremor) is the most common form of abnormal tremor. Although it may be mild and nonprogressive in some people, in others the tremor is slowly progressive, starting on one side of the body but eventually affecting both sides. Hand tremor is most common but the head, arms, voice, tongue, legs, and trunk may also be involved. Hand tremor may cause problems with purposeful movements such as eating, writing, sewing, or shaving. Head tremor may be seen as a “yes-yes” or “no-no” motion. Essential tremor may be accompanied by mild gait disturbance. Heightened emotion, stress, fever, physical exhaustion, or low blood sugar may trigger tremors or increase their severity. There may be mild degeneration in the certain parts of the cerebellum in persons with essential tremor. Onset is most common after age 40, although symptoms can appear at any age. Children of a parent who has essential tremor have up to a 50 percent chance of inheriting the condition. Essential tremor is not associated with any known pathology.

There is no definitive cure for essential tremor. Symptomatic drug therapy may include propranolol or other beta blockers and primidone, an anticonvulsant drug. Eliminating tremor “triggers” such as caffeine and other stimulants from the diet is often recommended. Physical and occupational therapy may help to reduce tremor and improve coordination and muscle control for some individuals. Deep brain stimulation uses a surgically implanted, battery-operated medical device called a neurostimulator to deliver electrical stimulation to targeted areas of the brain that control movement, temporarily blocking the nerve signals that cause tremor. Other surgical intervention is effective but may have side effects. U.S. patent application Ser. No. 16/356,517 describes use of gaboxadol to treat essential tremor. However, there remains a need for additional modalities for treatment of essential tremor.

Tourette syndrome (TS) is a neurological disorder characterized by repetitive, stereotyped, involuntary movements and vocalizations called tics. The first symptoms of TS are almost always noticed in childhood, usually appearing between the ages of 3 and 12. Some of the more common tics include eye blinking and other vision irregularities, throat clearing, grunting, facial grimacing, shoulder shrugging, and head or shoulder jerking. Perhaps the most dramatic and disabling tics are those that result in self-harm such as punching oneself, or vocal tics including coprolalia (uttering swear words) or echolalia (repeating the words or phrases of others). Medications may be administered to control some symptoms of TS. For example, typical and atypical neuroleptics including risperidone, ziprasidone, haloperidol, pimozide and fluphenazine may be utilized but can have long-term and short-term adverse effects. Antihypertensive agents such as clonidine and guanfacine are also used to treat tics.

Fragile X syndrome (FXS) may be the most common genetic cause of intellectual disability and the most common single-gene cause of autism. It is caused by mutations on the fragile X mental retardation gene (FMR1) and lack of fragile X mental retardation protein, which in turn, leads to decreased inhibition of translation of many synaptic proteins. The main efforts have focused on metabotropic glutamate receptor (mGluR) targeted treatments; however, investigation on the gamma-aminobutyric acid (GABA) system and its potential as a targeted treatment is less emphasized. Fragile X mouse models show decreased GABA subunit receptors, decreased synthesis of GABA, increased catabolism of GABA, and overall decreased GABAergic input in many regions of the brain. These symptoms are also observed in individuals with autism and other neurodevelopmental disorders, therefore targeted treatments for Fragile X syndrome are leading the way in the treatment of other neurodevelopmental syndromes and autism. Potential GABAergic treatments, such as riluzole, gaboxadol, tiagabine, and vigabatrin have been discussed. However, further studies are needed to determine the safety and efficacy of GABAergic treatments for Fragile X syndrome. Moreover, further studies in fragile X animal models are necessary to provide cumulative evidence in the efficacy and safety of gaboxadol. Lozano et al., Neuropsychiatr Dis Treat.,10: 1769-1779 (2014).

### SUMMARY

Pharmaceutical formulations containing gaboxadol or a pharmaceutically acceptable salt thereof and methods of treating essential tremors, Tourette syndrome or Fragile X syndrome are provided. Pharmaceutical formulations herein include transdermal formulations and modified release dosage forms. In embodiments, pharmaceutical formulations include about 0.05 mg to about 100 mg gaboxadol or a pharmaceutically acceptable salt thereof and are administered to a patient in need thereof. In embodiments, transdermal dosage forms contain a reservoir or matrix of gaboxadol monohydrate. In embodiments, transdermal dosage forms contain a reservoir or matrix of gaboxadol hydrochloride. In embodiments, a modified release dosage form includes an orally disintegrating dosage form. In embodiments, a modified release dosage form includes an extended release dosage form. In embodiments, a modified release dosage form includes a delayed release dosage form. In embodiments, a modified release dosage form includes a pulsatile release dosage form.

### DETAILED DESCRIPTION

Described herein are formulations and methods for treating essential tremor, Tourette syndrome or Fragile X syndrome by administering to a patient in need thereof a pharmaceutical formulation including gaboxadol or a pharmaceutically acceptable salt thereof. In embodiments, formulations and methods are described herein for treating essential tremor, Tourette syndrome or Fragile X syndrome by administering to a patient in need thereof a transdermal pharmaceutical formulation including gaboxadol or a pharmaceutically acceptable salt thereof. In embodiments, formulations and methods are described herein for treating essential tremor, Tourette syndrome or Fragile X syndrome by administering to a patient in need thereof a modified release pharmaceutical formulation including gaboxadol or a pharmaceutically acceptable salt thereof.

Many pharmaceutical products are administered as a fixed dose, at regular intervals, to achieve therapeutic efficacy. The duration of action is typically reflected by plasma half-life of the drug post administration. Gaboxadol has a relatively short half-life (t_{1/2}=1.5-2 h). Since efficacy is often dependent on rapid onset of action and sufficient exposure within the central nervous system, administration of CNS drugs with a short half-life may require frequent maintenance dosing.

Different clinical situations frequently require different therapeutic approaches. For example, treatment of an acute symptomatic episode may call for a dosage form which facilitates a rapid onset of action for fast relief of acute symptoms. For example, alleviating a sudden worsening of essential tremors, a sudden worsening of tics in the case of Tourette syndrome, or a sudden worsening of autistic behavior in Fragile X syndrome. Intravenous administration of a drug typically results in a more rapid onset of action than, for example, a conventional tablet or capsule formulation, which must be swallowed and disintegrated in the stomach before the drug can be absorbed. However, intravenous administration can be inconvenient in a non-clinical setting.

A modified release dosage form which provides rapid onset of action such as an orally disintegrating dosage form (“ODDF”), e.g., an orally disintegrating tablet (“ODT”) or orally disintegrating film (“ODF”) as described herein can advantageously release gaboxadol to the sublingual or buccal mucous membranes in the mouth (the oral mucosa). When gaboxadol comes into contact with the mucous membranes beneath the tongue and/or the cheek, it is absorbed directly into the bloodstream, thus bypassing the GI tract. This is because the connective tissue beneath the epithelium contains a rich network of capillaries into which the drug diffuses, thereby entering the venous circulation. In contrast, substances absorbed in the GI tract are subject to first-pass metabolism in the liver before entering the general circulation. Avoiding first pass metabolism can be preferable to conventional oral administration when rapid onset of action is desirable, since this route transports the gaboxadol directly to the brain, where it exerts it's extrasynaptic GABA_{A} agonism.

In other clinical situations such as those where symptoms are chronic, it may desirable to maintain a relatively constant sustained level of gaboxadol in the bloodstream leading to a sustained treatment of symptoms. In contrast to an ODDF containing gaboxadol where onset is rapid, but duration of action is not sustained due to the short half-life of gaboxadol, a modified dosage form herein having a sustained release profile provides a sustained therapeutic level of gaboxadol which provides a prolonged period of symptom relief without the need for repeated dosing throughout the day. As discussed in more detail below certain sustained relief dosage forms are administered orally and are absorbed in the GI tract where they undergo first pass metabolism.

Transdermal delivery of gaboxadol as described herein can provide sustained release profiles while avoiding first pass metabolism. Transdermal delivery is a painless method of delivering gaboxadol systemically by applying a formulation containing gaboxadol onto intact and healthy skin. The drug initially penetrates through the stratum corneum and then passes through the deeper epidermis. When the gaboxadol reaches the dermal layer, it becomes available for systemic absorption via dermal microcirculation. Transdermal delivery may have certain advantages over other routes of drug delivery. It can provide a non-invasive alternative to parenteral routes, thus circumventing issues such as needle phobia. A large surface area of skin and ease of access allows many placement options on the skin for transdermal absorption. Furthermore, the pharmacokinetic profile of transdermally administered gaboxadol may be more uniform with fewer peaks, thus minimizing the risk of toxic side effects. As with sustained release dosage forms, transdermal delivery can improve patient compliance due to the reduction of dosing frequencies and is also suitable for patients who are unconscious or vomiting, or those who rely on self-administration.

In embodiments, pharmaceutical formulations herein provide modified release of gaboxadol or a pharmaceutically acceptable salt thereof resulting in pharmacokinetic properties which include a Tₘₐₓ of 20 minutes or less. Accordingly, ODDF dosage forms are described that provide a rapid onset of action. In embodiments, pharmaceutical formulations having modified release profiles provide pharmacokinetic properties which result in both rapid onset and sustained duration of action. In embodiments, pharmaceutical formulations having modified release profiles provide pharmacokinetic properties which result in both rapid onset and extended release. In embodiments, pharmaceutical formulations having modified release profiles provide pharmacokinetic properties which result in both rapid onset and delayed release. In embodiments, pharmaceutical formulations having modified release profiles provide pharmacokinetic properties which result in both rapid onset and extended release which is pulsatile in nature. In embodiments, pharmaceutical formulations having modified release profiles provide pharmacokinetic properties which result in both rapid onset and delayed release which is pulsatile in nature. In embodiments, pharmaceutical formulations having modified release profiles provide pharmacokinetic properties which result in a combination of rapid onset, delayed release and sustained duration of action. In embodiments, pharmaceutical formulations having modified release profiles provide pharmacokinetic properties which result in a combination of rapid onset, delayed release and sustained duration of action which is pulsatile in nature.

Conventional (or unmodified) release oral dosage forms such as tablets or capsules typically release medications into the stomach or intestines as the tablet or capsule shell dissolves. The pattern of drug release from modified release (MR) dosage forms is deliberately changed from that of a conventional dosage form to achieve a desired therapeutic objective and/or better patient compliance. Types of MR drug products include, 1) orally disintegrating dosage forms (ODDFs) which provide immediate release, 2) extended release dosage forms, 3) delayed release dosage forms (e.g., enteric coated), 4) pulsatile release dosage forms, and 5) combinations of the foregoing.

In embodiments, pharmaceutical formulations herein provide immediate release of gaboxadol or a pharmaceutically acceptable salt thereof resulting in pharmacokinetic properties which include a Tₘₐₓ of 20 minutes or less. In embodiments, pharmaceutical formulations herein provide a Tₘₐₓ of 20 minutes or less, a Tₘₐₓ of 19 minutes or less, a Tₘₐₓ of 18 minutes or less, a Tₘₐₓ of 17 minutes or less, a Tₘₐₓ of 16 minutes or less, a Tₘₐₓ of 15 minutes or less, a Tₘₐₓ of 14 minutes or less, a Tₘₐₓ of 13 minutes or less, a Tₘₐₓ of 12 minutes or less, a Tₘₐₓ of 11 minutes or less, a Tₘₐₓ of 10 minutes or less, a Tₘₐₓ of 9 minutes or less, a Tₘₐₓ of 8 minutes or less, a Tₘₐₓ of 7 minutes or less, a Tₘₐₓ of 6 minutes or less, or a Tₘₐₓ of 5 minutes or less. Such pharmaceutical formulations include ODDFs such as orally disintegrating tablets (ODTs) or orally disintegrating films (ODFs).

An ODDF is a solid dosage form containing a medicinal substance or active ingredient which disintegrates rapidly, usually within a matter of seconds when placed upon the tongue, sublingually or buccally. The disintegration time for ODDFs generally range from one or two seconds to about a minute. ODDFs are designed to disintegrate or dissolve rapidly on contact with saliva. This mode of administration can be beneficial to people who may have problems swallowing tablets whether it be from physical infirmity or psychiatric in nature. Patients with essential tremors, Tourette syndrome or Fragile X syndrome may exhibit such behavior. In addition, ODDFs herein provide a rapid onset of action which can provide rapid alleviation or cessation of symptoms associated with essential tremors, Tourette syndrome or Fragile X syndrome, respectively. In embodiments, when administered to an oral cavity, an ODDF herein disintegrates in less than one minute, less than 55 seconds, less than 50 seconds, less than 45 seconds, less than 40 seconds, less than 35 seconds, less than 30 seconds, less than 25 seconds, less than 20 seconds, less than 15 seconds, less than 10 seconds, or less than 5 seconds.

An ODT is a solid dosage form containing a medicinal substance or active ingredient which disintegrates rapidly, usually within a matter of seconds when placed upon the tongue, sublingually or buccally. The disintegration time for ODTs generally ranges from several seconds to about a minute. ODTs are designed to disintegrate or dissolve rapidly on contact with saliva, thus eliminating the need to chew the tablet, swallow the intact tablet, or take the tablet with liquids. As with ODDF s in general, this mode of administration can be beneficial to people who may have problems swallowing tablets whether it be from physical infirmity or psychiatric in nature. Patients with essential tremors, Tourette syndrome or Fragile X syndrome may exhibit such behavior. In addition, ODTs herein provide a rapid onset of action which can result in a rapid alleviation or cessation of symptoms associated with essential tremors, Tourette syndrome or Fragile X syndrome, respectively. In embodiments, an ODT herein disintegrates in less than one minute, less than 55 seconds, less than 50 seconds, less than 45 seconds, less than 40 seconds, less than 35 seconds, less than 30 seconds, less than 25 seconds, less than 20 seconds, less than 15 seconds, less than 10 seconds, or less than 5 seconds, based upon, e.g., the United States Pharmacopeia (USP) disintegration test method set forth at section 701, Revision Bulletin Official Aug. 1, 2008.

In embodiments, the fast dissolving property of the ODTs requires quick ingress of water into the tablet matrix. This may be accomplished by maximizing the porous structure of the tablet, incorporation of suitable disintegrating agents and use of highly water-soluble excipients in the formulation. Excipients used in ODTs typically contain at least one superdisintegrant (which can have a mechanism of wicking, swelling or both), a diluent, a lubricant and optionally a swelling agent, sweeteners and flavorings. See, e.g., Nagar et al., Journal of Applied Pharmaceutical Science, 2011;01(04):35-45, incorporated herein by reference. Superdisintegrants can be classified as synthetic, natural and co-processed. In this context synthetic superdisintegrants can be exemplified by sodium starch glycolate, croscarmellose sodium, cross-linked polyvinylpyrrolidone, low-substituted hydroxypropyl cellulose, microcrystalline cellulose, partially pregelatinized starch, cross-linked alginic acid and modified resin. Natural superdisintegrants can be processed mucilages and gums are obtained from plants and can be exemplified by Lepidium sativum seed mucilage, banana powder, gellan gum, locust bean gum, xanthan gum, guar gum, gum karaya, cassia fistula seed gum, mangifera indica gum, carrageenan, agar from Gelidium amansii and other red algaes, soy polysaccharide and chitosan. Diluents can include, e.g., mannitol, sorbitol, xylitol, calcium carbonate, magnesium carbonate, calcium sulfate, magnesium tri silicate and the like. Lubricants can include, e.g., magnesium stearate and the like. Those skilled in the art are familiar with ODT manufacturing techniques.

Other ODDFs which may be used herein include rapidly dissolving films which are thin oral strips that release medication such as gaboxadol or a pharmaceutically acceptable salt thereof quickly after administration to the oral cavity. The film is placed on a patient's tongue, sublingually, bucally, or on any other mucosal surface and is instantly wet by saliva whereupon the film rapidly hydrates and dissolves to release the medication. See. e.g., Chaturvedi et al., Curr Drug Deliv. 2011 July; 8(4):373-80. Fastcaps are a rapidly disintegrating drug delivery system based on gelatin capsules. In contrast to conventional hard gelatin capsules, fastcaps consist of a gelation of low bloom strength and various additives to improve the mechanical and dissolution properties of the capsule shell. Fastcaps are also referred to herein as orally disintegrating capsules. See, e.g., Ciper and Bodmeier, Int J Pharm. 2005 Oct. 13; 303(1-2):62-71. Freeze dried (lyophilized) wafers (also referred to herein as orally disintegrating wafers) are rapidly disintegrating, thin matrixes that contain a medicinal agent. The wafer or film disintegrates rapidly in the oral cavity and releases drug which dissolves or disperses in the saliva. See, e.g., Boateng et al., Int J Pharm. 2010 Apr. 15; 389(1-2):24-31. Those skilled in the art are familiar with various techniques utilized to manufacture ODDFs such as freeze drying, spray drying, phase transition processing, melt granulation, sublimation, mass extrusion, cotton candy processing, direct compression, etc. See, e.g., Nagar et al., supra.

When administered, ODDFs containing gaboxadol or a pharmaceutically acceptable salt thereof disintegrate rapidly to release the drug, which dissolves or disperses in the saliva. The drug may be absorbed in the oral cavity, e.g., sublingually, buccally, from the pharynx and esophagus or from other sections of gastrointestinal tract as the saliva travels down. In such cases, bioavailability can be significantly greater than that observed from conventional tablet dosage forms which travel to the stomach or intestines where drug can be released.

ODDFs herein provide a Tₘₐₓ of 20 minutes or less, a Tₘₐₓ of 19 minutes or less, a Tₘₐₓ of 18 minutes or less, a Tₘₐₓ of 17 minutes or less, a Tₘₐₓ of 16 minutes or less, a Tₘₐₓ of 15 minutes or less, a Tₘₐₓ of 14 minutes or less, a Tₘₐₓ of 13 minutes or less, a Tₘₐₓ of 12 minutes or less, a Tₘₐₓ of 11 minutes or less, a Tₘₐₓ of 10 minutes or less, a Tₘₐₓ of 9 minutes or less, a Tₘₐₓ of 8 minutes or less, a Tₘₐₓ of 7 minutes or less, a Tₘₐₓ of 6 minutes or less, or a Tₘₐₓ of 5 minutes or less. In embodiments, the amount of gaboxadol or pharmaceutically acceptable salt thereof within the patient about 4 hours after administration of the pharmaceutical formulation is between about 65% to about 85% less than the administered dose. In embodiments, the amount of gaboxadol or pharmaceutically acceptable salt thereof within the patient about 4 hours after administration of the pharmaceutical formulation is less than 65% 70%, 75%, 80%, or 85% of the administered dose.

In embodiments, ODDFs herein provide an in vivo plasma profile having C. less than about 2500 ng/ml, 2000 ng/ml, 1750 ng/ml, 1500 ng/ml, 1250 ng/ml, 1000 ng/ml, 750 ng/ml, 500 ng/ml, 450 ng/ml, 400 ng/ml, 350 ng/ml, 300 ng/ml, 250 ng/ml, 200 ng/ml, 150 ng/ml, 100 ng/ml, 50 ng/ml or 25 ng/ml. In embodiments, ODDFs herein provide an in vivo plasma profile having a AUC_{0-∞} of less than about, e.g., 900 ng·hr/ml, 850 ng·hr/ml, 800 ng·hr/ml, 750 ng·hr/ml, or 700 ng·hr/ml 650 ng·hr/ml, 600 ng·hr/ml, 550 ng·hr/ml, 500 ng·hr/ml, or 450 ng·hr/ml. In embodiments, ODDFs herein provide an in vivo plasma profile having a AUC_{0-∞} of less than about, e.g., 400 ng·hr/ml, 350 ng·hr/ml, 300 ng·hr/ml, 250 ng·hr/ml, or 200 ng·hr/ml. In embodiments, ODDFs herein provide an in vivo plasma profile having a AUC₀₋₂₈ of less than about, e.g., 150 ng·hr/ml, 100 ng·hr/ml, 75 ng·hr/ml, or 50 ng·hr/ml.

In embodiments, pharmaceutical formulations having modified release profiles provide pharmacokinetic properties which result in both rapid onset and sustained duration of action. Such pharmaceutical formulations include an immediate release aspect and an extended release aspect. Immediate release aspects are discussed above in connection with ODDFs. Extended release dosage forms (ERDFs) have an extended release profiles and are those that allow a reduction in dosing frequency as compared to that presented by a conventional dosage form, e.g., a solution or unmodified release dosage form. ERDFs provide a sustained duration of action of a drug. In embodiments, modified release dosage forms herein may incorporate an ODDF aspect to provide immediate release of a loading dose and then an ERDF aspect that provides prolonged delivery to maintain drug levels in the blood within a desired therapeutic range for a desirable period of time in excess of the activity resulting from a single dose of the drug. In embodiments, the ODDF aspect releases the drug immediately and the ERDF aspect thereafter provides continuous release of drug for sustained action. In embodiments, ERDFs are not combined with an ODDF aspect and can be administered as a solitary dosage form.

In embodiments, the immediate release aspect achieves a Tₘₐₓ of 20 minutes or less, a Tₘₐₓ of 19 minutes or less, a Tₘₐₓ of 18 minutes or less, a Tₘₐₓ of 17 minutes or less, a Tₘₐₓ of 16 minutes or less, a Tₘₐₓ of 15 minutes or less, a Tₘₐₓ of 14 minutes or less, a Tₘₐₓ of 13 minutes or less, a Tₘₐₓ of 12 minutes or less, a Tₘₐₓ of 11 minutes or less, a Tₘₐₓ of 10 minutes or less, a Tₘₐₓ of 9 minutes or less, a Tₘₐₓ of 8 minutes or less, a Tₘₐₓ of 7 minutes or less, a Tₘₐₓ of 6 minutes or less, or a Tₘₐₓ of 5 minutes or less. In embodiments, the extended release aspect provides an amount of gaboxadol or pharmaceutically acceptable salt thereof within the patient at about 4 or more hours after administration of the pharmaceutical formulation between about 50% to about 100% of the initially administered ODDF dose. In embodiments, the amount of gaboxadol or pharmaceutically acceptable salt thereof within the patient about 4 hours after administration of the pharmaceutical formulation is more than 50%, 55%, 60%, 65%, 70%, 75%, 80%, 85%, 90%, 95%, 100%, 105%, or 110% of the initially administered ODDF dose. In embodiments, the extended release aspect provides an amount of gaboxadol or pharmaceutically acceptable salt thereof within the patient at about 6 or more hours after administration of the pharmaceutical formulation between about 50% to about 110% of the initially administered ODDF dose. In embodiments, the amount of gaboxadol or pharmaceutically acceptable salt thereof within the patient about 6 hours after administration of the pharmaceutical formulation is more than 50%, 55%, 60%, 65%, 70%, 75%, 80%, 85%, 90%, 95%, 100%, 105%, or 110% of the initially administered ODDF dose. In embodiments, the extended release aspect provides an amount of gaboxadol or pharmaceutically acceptable salt thereof within the patient at about 8 or more hours after administration of the pharmaceutical formulation between about 50% to about 110% of the initially administered ODDF dose. In embodiments, the amount of gaboxadol or pharmaceutically acceptable salt thereof within the patient about 8 hours after administration of the pharmaceutical formulation is more than 50%, 55%, 60%, 65%, 70%, 75%, 80%, 85%, 90%, 95%, 100%, 105%, or 110% of the initially administered ODDF dose. In embodiments, the extended release aspect provides an amount of gaboxadol or pharmaceutically acceptable salt thereof within the patient at about 10 or more hours after administration of the pharmaceutical formulation between about 50% to about 110% of the initially administered ODDF dose. In embodiments, the amount of gaboxadol or pharmaceutically acceptable salt thereof within the patient about 10 hours after administration of the pharmaceutical formulation is more than 50%, 55%, 60%, 65%, 70%, 75%, 80%, 85%, 90%, 95%, 100%, 105%, or 110% of the initially administered ODDF dose. In embodiments, the extended release aspect provides an amount of gaboxadol or pharmaceutically acceptable salt thereof within the patient at about 12 or more hours after administration of the pharmaceutical formulation between about 50% to about 110% of the initially administered ODDF dose. In embodiments, the amount of gaboxadol or pharmaceutically acceptable salt thereof within the patient about 12 hours after administration of the pharmaceutical formulation is more than 50%, 55%, 60%, 65%, 70%, 75%, 80%, 85%, 90%, 95%, 100%, 105%, or 110% of the initially administered ODDF dose.

In embodiments, an ODDF is applied as a coating or band over an ERDF, or as a layer adjacent to an ERDF, to allow direct exposure of the ODDF to the oral cavity and consequent disintegration of the ODDF. In embodiments, the ODDF and the ERDF can be mixed in a chewable resin, e.g., gum. Those skilled in the art are familiar with techniques for applying coatings, bands and layers to fabricate pharmaceutical dosage forms.

Suitable formulations which provide extended release profiles are well-known in the art. For example, coated slow release beads or granules (“beads” and “granules” are used interchangeably herein) in which, e.g., gaboxadol or a pharmaceutically acceptable salt thereof is applied to beads, e.g., confectioners nonpareil beads, and then coated with conventional release retarding materials such as waxes, enteric coatings and the like. In embodiments, beads can be formed in which gaboxadol or pharmaceutically acceptable salt thereof is mixed with a material to provide a mass from which the drug leaches out. In embodiments, the beads may be engineered to provide different rates of release by varying characteristics of the coating or mass, e.g., thickness, porosity, using different materials, etc. Beads having different rates of release may be combined into a single dosage form to provide variable or continuous release. The beads can be contained in capsules or compressed into tablets. In embodiments, the ODDF is applied as a coating, a layer or a band to a capsule or tablet. In embodiments, slow release cores which are incorporated into tablets or capsules can also provide extended release profiles. For example, gaboxadol or a pharmaceutically acceptable salt thereof can be mixed in a substance or a mixture of substances non-absorbable from the gastrointestinal tract but capable of slow dissolution or loss of drug by leaching, and an outer ODDF layer which is applied to the core by, e.g., compression or spraying. In embodiments, extended release profiles may be provided by multiple layer tablets, each layer having different release properties. Multilayer tableting machines allow incorporation into one tablet of two or more separate layers which may be made to release gaboxadol or a pharmaceutically acceptable salt thereof at different rates. For example, one or more outer layers may be an ODDF, and each other layer an ERDF that exhibits different release rates. In embodiments, gaboxadol or a pharmaceutically acceptable salt thereof is incorporated into porous inert carriers that provide extended release profiles. In embodiments, the porous inert carriers incorporate channels or passages from which the drug diffuses into surrounding fluids. In embodiments, gaboxadol or a pharmaceutically acceptable salt thereof is incorporated into an ion-exchange resin to provide an extended release profile. Prolonged action results from a predetermined rate of release of the drug from the resin when the drug-resin complex contacts gastrointestinal fluids and the ionic constituents dissolved therein. In embodiments, membranes are utilized to control rate of release from drug containing reservoirs. In embodiments, liquid preparations may also be utilized to provide an extended release profile. For example, a liquid preparation consisting of solid particles dispersed throughout a liquid phase in which the particles are not soluble. The suspension is formulated to allow at least a reduction in dosing frequency as compared to that drug presented as a conventional dosage form (e.g., as a solution or a prompt drug-releasing, conventional solid dosage form). For example, a suspension of ion-exchange resin constituents or microbeads.

In embodiments, absorbable or non-absorbable polymers may be utilized to form ERDFs. Various ERDFs including those discussed above and others that can be utilizable herein are known to those with skill in the art. See, e.g., Fu and Kao,Expert Opin Drug Deliv. 2010 April; 7(4): 429-444.

In embodiments, modified dosage forms herein encompass delayed release dosage forms having delayed release profiles. Delayed release dosage forms can include delayed release tablets or delayed release capsules. A delayed release tablet is a solid dosage form which releases a drug (or drugs) such as gaboxadol or a pharmaceutically acceptable salt thereof at a time other than promptly after administration. A delayed release capsule is a solid dosage form in which the drug is enclosed within either a hard or soft soluble container made from a suitable form of gelatin, and which releases a drug (or drugs) at a time other than promptly after administration. For example, with respect to tablets or capsules, enteric-coated articles are examples of delayed release dosage forms. In embodiments, a delayed release tablet is a solid dosage form containing a conglomerate of medicinal particles that releases a drug (or drugs) at a time other than promptly after administration. In embodiments, the conglomerate of medicinal particles are covered with a coating which delays release of the drug. In embodiments, a delayed release capsule is a solid dosage form containing a conglomerate of medicinal particles that releases a drug (or drugs) at a time other than promptly after administration. In embodiments, the conglomerate of medicinal particles are covered with a coating which delays release of the drug.

In embodiments, ODDFs with a delayed release formulation aspect are provided that are solid dosage forms containing medicinal substances which disintegrate rapidly, usually within a matter of seconds, when placed upon the tongue, but which also releases a drug (or drugs) at a time other than promptly after administration. Accordingly, in embodiments, modified release dosage forms herein incorporate an ODDF aspect to provide immediate release of a loading dose and then an a delayed release formulation aspect that provides a period in which there is no drug delivery followed by a period of drug delivery to provide drug levels in the blood within a desired therapeutic range for a desirable period of time in excess of the activity resulting from a single dose of the drug. In embodiments, the ODDF aspect releases the drug immediately and then, after a period of delay, a delayed release formulation aspect thereafter provides a single release of drug to provide an additional period of activity. In embodiments, the ODDF aspect releases the drug immediately and then, after a period of delay, a delayed release formulation aspect thereafter provides a continuous release of drug for sustained action.

In embodiments, the immediate release aspect of a ODDF with a delayed release aspect achieves a Tₘₐₓ of 20 minutes or less, a Tₘₐₓ of 19 minutes or less, a Tₘₐₓ of 18 minutes or less, a Tₘₐₓ of 17 minutes or less, a Tₘₐₓ of 16 minutes or less, a Tₘₐₓ of 15 minutes or less, a Tₘₐₓ of 14 minutes or less, a Tₘₐₓ of 13 minutes or less, a Tₘₐₓ of 12 minutes or less, a Tₘₐₓ of 11 minutes or less, a Tₘₐₓ of 10 minutes or less, a Tₘₐₓ of 9 minutes or less, a Tₘₐₓ of 8 minutes or less, a Tₘₐₓ of 7 minutes or less, a Tₘₐₓ of 6 minutes or less, or a Tₘₐₓ of 5 minutes or less. In embodiments, the delayed release aspect provides an amount of gaboxadol or pharmaceutically acceptable salt thereof within the patient at about 1, 2, 3 or 4 or more hours after administration of the pharmaceutical formulation between about 50% to about 110% of the initially administered ODDF dose. In embodiments, the amount of gaboxadol or pharmaceutically acceptable salt thereof within the patient about 1, 2, 3 or 4 hours after administration of the pharmaceutical formulation is more than 50%, 55%, 60%, 65%, 70%, 75%, 80%, 85%, 90%, 95%, 100%, 105%, or 110% of the initially administered ODDF dose. In embodiments, the delayed release formulation aspect provides an amount of gaboxadol or pharmaceutically acceptable salt thereof within the patient at about 6 or more hours after administration of the pharmaceutical formulation between about 50% to about 110% of the initially administered ODDF dose. In embodiments, the amount of gaboxadol or pharmaceutically acceptable salt thereof within the patient about 6 hours after administration of the pharmaceutical formulation is more than 50%, 55%, 60%, 65%, 70%, 75%, 80%, 85%, 90%, 95%, 100%, 105%, or 110% of the initially administered ODDF dose. In embodiments, the delayed release formulation aspect provides an amount of gaboxadol or pharmaceutically acceptable salt thereof within the patient at about 8 or more hours after administration of the pharmaceutical formulation between about 50% to about 110% of the initially administered ODDF dose. In embodiments, the amount of gaboxadol or pharmaceutically acceptable salt thereof within the patient about 8 hours after administration of the pharmaceutical formulation is more than 50%, 55%, 60%, 65%, 70%, 75%, 80%, 85%, 90%, 95%, 100%, 105%, or 110% of the initially administered ODDF dose. In embodiments, the delayed release formulation aspect provides an amount of gaboxadol or pharmaceutically acceptable salt thereof within the patient at about 10 or more hours after administration of the pharmaceutical formulation between about 50% to about 110% of the initially administered ODDF dose. In embodiments, the amount of gaboxadol or pharmaceutically acceptable salt thereof within the patient about 10 hours after administration of the pharmaceutical formulation is more than 50%, 55%, 60%, 65%, 70%, 75%, 80%, 85%, 90%, 95%, 100%, 105%, or 110% of the initially administered ODDF dose. In embodiments, the delayed release formulation aspect provides an amount of gaboxadol or pharmaceutically acceptable salt thereof within the patient at about 12 or more hours after administration of the pharmaceutical formulation between about 50% to about 110% of the initially administered ODDF dose. In embodiments, the amount of gaboxadol or pharmaceutically acceptable salt thereof within the patient about 12 hours after administration of the pharmaceutical formulation is more than 50%, 55%, 60%, 65%, 70%, 75%, 80%, 85%, 90%, 95%, 100%, 105%, or 110% of the initially administered ODDF dose.

Delayed release dosage forms are known to those skilled in the art. For example, coated delayed release beads or granules (“beads” and “granules” are used interchangeably herein) in which, e.g., gaboxadol or a pharmaceutically acceptable salt thereof is applied to beads, e.g., confectioners nonpareil beads, and then coated with conventional release delaying materials such as waxes, enteric coatings and the like. In embodiments, beads can be formed in which gaboxadol or pharmaceutically acceptable salt thereof is mixed with a material to provide a mass from which the drug leaches out. In embodiments, the beads may be engineered to provide different rates of release by varying characteristics of the coating or mass, e.g., thickness, porosity, using different materials, etc. In embodiments, enteric coated granules of gaboxadol or a pharmaceutically acceptable salt thereof can be contained in an enterically coated capsule or tablet which releases the granules in the small intestine. In embodiments, the granules have a coating which remains intact until the coated granules reach at least the ileum and thereafter provide a delayed release of the drug in the colon. Suitable enteric coating materials are well known in the art, e.g., Eudragit® coatings such methacrylic acid and methyl methacrylate polymers and others. The granules can be contained in capsules or compressed into tablets. In embodiments, the ODDF is applied as a coating, a layer or a band to the capsule or tablet. In embodiments, delayed release cores which are incorporated into tablets or capsules can also provide delayed release profiles. For example, gaboxadol or a pharmaceutically acceptable salt thereof can be mixed in a substance or a mixture of substances non-absorbable from the gastrointestinal tract but capable of slow dissolution or loss of drug by leaching, and an outer ODDF layer which is applied to the core by, e.g., compression or spraying. In embodiments, delayed release profiles may be provided by multiple layer tablets, each layer having different release properties. Multilayer tableting machines allow incorporation into one tablet of two or more separate layers which may be made to release gaboxadol or a pharmaceutically acceptable salt thereof at different rates after a period of delay. For example, one or more outer layers may be an ODDF, and each other layer a delayed release dosage form that exhibits different release rates. In embodiments, gaboxadol or a pharmaceutically acceptable salt thereof is incorporated into porous inert carriers that provide delayed release profiles. In embodiments, the porous inert carriers incorporate channels or passages from which the drug diffuses into surrounding fluids. In embodiments, gaboxadol or a pharmaceutically acceptable salt thereof is incorporated into an ion-exchange resin to provide a delayed release profile. Delayed action may result from a predetermined rate of release of the drug from the resin when the drug-resin complex contacts gastrointestinal fluids and the ionic constituents dissolved therein. In embodiments, membranes are utilized to control rate of release from drug containing reservoirs. In embodiments, liquid preparations may also be utilized to provide a delayed release profile. For example, a liquid preparation consisting of solid particles dispersed throughout a liquid phase in which the particles are not soluble. The suspension is formulated to allow at least a reduction in dosing frequency as compared to that drug presented as a conventional dosage form (e.g., as a solution or a prompt drug-releasing, conventional solid dosage form). For example, a suspension of ion-exchange resin constituents or microbeads.

In embodiments, an ODDF is applied as a coating or band over a delayed release dosage form, or as a layer adjacent to a delayed release dosage form, to allow direct exposure of the ODDF to the oral cavity and consequent disintegration of the ODDF. In embodiments, the ODDF and a delayed release dosage form can be mixed in a chewable resin, e.g., gum. Those skilled in the art are familiar with techniques for applying coatings, bands and layers to fabricate pharmaceutical dosage forms.

In embodiments, modified release pharmaceutical formulations herein include pulsatile release dosage formulations (PRDFs). Pulsatile drug release involves rapid release of defined or discrete amounts of a drug (or drugs) such as gaboxadol or a pharmaceutically acceptable salt thereof after a lag time following an initial release of drug. In embodiments, PRDFs can provide a single pulse. In embodiments, PRDFs can provide multiple pulses over time. Various PRDFs are known to those with skill in the art.

In embodiments, a PRDF can be a capsule. In embodiments, release after a lag time is provided by a system that uses osmotic pressure to cause release of a plug. In this system, gaboxadol or a pharmaceutically acceptable salt thereof is contained in an insoluble capsule shell sealed by an osmotically responsive plug, e.g., a hydrogel, which is pushed away by swelling or erosion. When the seal is broken the drug is released as a pulse from the capsule body. Contact with gastrointestinal fluid or dissolution medium causes the plug to swell, either pushing itself out of the capsule or causing the capsule to rupture after the lag-time. Position & dimensions of the plug can control lag-time. For rapid release of drug effervescent or disintegrating agents may be added. Effervescent materials can cause an increase in pressure thus aiding or causing expulsion of the plug. Examples of suitable plug material may be swellable materials coated with permeable polymer (polymethacrylates), erodible compressed polymer (HPMC, polyvinyl alcohol), congealed melted polymer (glyceryl monooleate), and enzymatically controlled erodible polymers such as pectin. In embodiments, an insoluble capsule contains multiple drug compartments separated by osmotically activated plugs. When a first plug is exposed to the environmental fluids, the first compartment opens, drug is released and the adjacent plug is exposed. The process continues until no sealed compartment are left. Lag time between pulses can be further controlled by varying the thickness of the plug and the properties of the materials from which the plug is made. More hygroscopic materials will absorb fluid faster and will swell faster. In embodiments, a membrane may be substituted for the plug. If effervescent materials are included in one or more compartments, fluids pass through the membrane by osmosis and the effervescent action and pressure increase causes the membrane to rupture, thereby releasing the drug. In embodiments, the membrane(s) are erodible and dissolve to release the contents of the compartment(s). Varying the thickness, porosity and properties of materials of the membrane can allow further control of lag time between pulses. In embodiments, a PRDF can be a tablet. In embodiments, single pulse tablets involve a core containing gaboxadol or a pharmaceutically acceptable salt thereof surrounded by one or more layers of swellable, rupturable coatings. In embodiments, a rupturable coating surrounds a swellable layer. As the swellable layer expands, it causes the rupturable coating to rupture, thereby releasing the drug from the core. Swellable materials such as hydrogels are well known. In embodiments, an inner swelling layer can contain a superdisintegrant, e.g., croscarmellose sodium, and an outer rupturable layer can be made of a polymeric porous materials such as polyethylene oxides, ethylcellulose and the like. Porous film coats of sucrose may also be suitable. In embodiments, multiple pulse tablets incorporate multiple layers surrounding a core. As a first outermost layer erodes and releases the drug contained within the layer, an underlying layer is exposed, thus releasing drug after a predetermined lag time. The process repeats until the innermost core is exposed.

In embodiments, PRDFs can incorporate ODDFs that are solid dosage forms containing medicinal substances which disintegrate rapidly, usually within a matter of seconds, when placed upon the tongue, but which also releases a drug (or drugs) in pulsatile fashion. Accordingly, in embodiments, modified release dosage forms herein incorporate an ODDF aspect to provide immediate release of a loading dose and a PRDF aspect that provides a period in which there is no drug delivery (lag time) followed by pulsatile drug delivery to provide drug levels in the blood within a desired therapeutic range for a desirable period of time in excess of the activity resulting from a single dose of the drug. In embodiments, the ODDF aspect releases the drug immediately and then, after a period of delay, the PRDF aspect thereafter provides a single pulse release of drug to provide an additional period of activity. In embodiments, the ODDF aspect releases the drug immediately and then, after a period of delay, the PRFD aspect thereafter provides multiple pulsatile release of drug for prolonged therapeutic effect.

In embodiments, the immediate release aspect of a ODDF with a PRDF aspect achieves a Tₘₐₓ of 20 minutes or less, a Tₘₐₓ of 19 minutes or less, a Tₘₐₓ of 18 minutes or less, a Tₘₐₓ of 17 minutes or less, a Tₘₐₓ of 16 minutes or less, a Tₘₐₓ of 15 minutes or less, a Tₘₐₓ of 14 minutes or less, a Tₘₐₓ of 13 minutes or less, a Tₘₐₓ of 12 minutes or less, a Tₘₐₓ of 11 minutes or less, a Tₘₐₓ of 10 minutes or less, a Tₘₐₓ of 9 minutes or less, a Tₘₐₓ of 8 minutes or less, a Tₘₐₓ of 7 minutes or less, a Tₘₐₓ of 6 minutes or less, or a Tₘₐₓ of 5 minutes or less. In embodiments, a PRDF aspect provides an amount of gaboxadol or pharmaceutically acceptable salt thereof within the patient at about 0.5, 1, 2, 3 or 4 or more hours after administration of the pharmaceutical formulation between about 50% to about 110% of the initially administered ODDF dose. In embodiments, the amount of gaboxadol or pharmaceutically acceptable salt thereof within the patient about 0.5, 1, 2, 3 or 4 hours after administration of the pharmaceutical formulation is more than 50%, 55%, 60%, 65%, 70%, 75%, 80%, 85%, 90%, 95%, 100%, 105%, or 110% of the initially administered ODDF dose. In embodiments, a PRDF aspect provides an amount of gaboxadol or pharmaceutically acceptable salt thereof within the patient at about 6 or more hours after administration of the pharmaceutical formulation between about 50% to about 110% of the initially administered ODDF dose. In embodiments, the amount of gaboxadol or pharmaceutically acceptable salt thereof within the patient about 6 hours after administration of the pharmaceutical formulation is more than 50%, 55%, 60%, 65%, 70%, 75%, 80%, 85%, 90%, 95%, 100%, 105%, or 110% of the initially administered ODDF dose. In embodiments, a PRDF aspect provides an amount of gaboxadol or pharmaceutically acceptable salt thereof within the patient at about 8 or more hours after administration of the pharmaceutical formulation between about 50% to about 110% of the initially administered ODDF dose. In embodiments, the amount of gaboxadol or pharmaceutically acceptable salt thereof within the patient about 8 hours after administration of the pharmaceutical formulation is more than 50%, 55%, 60%, 65%, 70%, 75%, 80%, 85%, 90%, 95%, 100%, 105%, or 110% of the initially administered ODDF dose. In embodiments, a PRDF aspect provides an amount of gaboxadol or pharmaceutically acceptable salt thereof within the patient at about 10 or more hours after administration of the pharmaceutical formulation between about 50% to about 110% of the initially administered ODDF dose. In embodiments, the amount of gaboxadol or pharmaceutically acceptable salt thereof within the patient about 10 hours after administration of the pharmaceutical formulation is more than 50%, 55%, 60%, 65%, 70%, 75%, 80%, 85%, 90%, 95%, 100%, 105%, or 110% of the initially administered ODDF dose. In embodiments, a PRDF aspect provides an amount of gaboxadol or pharmaceutically acceptable salt thereof within the patient at about 12 or more hours after administration of the pharmaceutical formulation between about 50% to about 110% of the initially administered ODDF dose. In embodiments, the amount of gaboxadol or pharmaceutically acceptable salt thereof within the patient about 12 hours after administration of the pharmaceutical formulation is more than 50%, 55%, 60%, 65%, 70%, 75%, 80%, 85%, 90%, 95%, 100%, 105%, or 110% of the initially administered ODDF dose. In embodiments, the PRDF delivers one pulse in accordance with the above amounts. In embodiments, the PRDF delivers two pulses in accordance with the above amounts. In embodiments, the PRDF delivers three pulses in accordance with the above amounts. In embodiments, the PRDF delivers four pulses in accordance with the above amounts. In embodiments, the PRDF delivers five pulses in accordance with the above amounts. In embodiments, the PRDF delivers six pulses in accordance with the above amounts. In embodiments, the PRDF delivers seven pulses in accordance with the above amounts. In embodiments, the PRDF delivers eight pulses in accordance with the above amounts. In embodiments, the PRDF delivers nine pulses in accordance with the above amounts. The pulses may be provided in intervals separated by 0.25 h, 0.5 h, 0.75 h, 1 h, 1.25 h, 1.5 h, 1.75 h, 2, h, 2.25 h, 2.5 h, 2.75 h, 3 h, 3.25 h, 3.5 h, 3.75 h, 4 h, 4.25 h, 4.5 h, 4.75 h, 5 h, 5.5 h, 6 h, 7 h, 8 h, 9 h, 10 h, 11 h, or 12 h. In embodiments the amount of gaboxadol or a pharmaceutically acceptable salt thereof released with each pulse may vary.

In embodiments, an ODDF is applied as a coating or band over a PRDF, or as a layer adjacent to a PRDF, to allow direct exposure of the ODDF to the oral cavity and consequent disintegration of the ODDF. In embodiments, the ODDF and a PRDF can be mixed in a chewable resin, e.g., gum. Those skilled in the art are familiar with techniques for applying coatings, bands and layers to fabricate pharmaceutical dosage forms.

In embodiments, transdermal pharmaceutical formulations are provided for treatment of essential tremors, Tourette syndrome or Fragile X syndrome. Transdermal formulations may encompass dosage forms of gels, ointments, lotions, sprays, or patches. Transdermal formulations such as patches rely for their effect, on delivery of a known flux of drug to the skin for a prolonged period of time, generally a day, several days, or a week. Two mechanisms may be used to regulate drug flux: either the drug is contained within a drug reservoir, which is separated from the skin of the wearer by a synthetic membrane, through which the drug diffuses; or the drug is held dissolved or suspended in a polymer matrix, through which the drug diffuses to the skin. In embodiments, transdermal pharmaceutical formulations herein are formulated to provide maximum thermodynamic driving force for passive diffusion across the skin which is saturated with sufficient payload of gaboxadol to insure delivery across the skin. In delivery systems involving transdermal patches, gaboxadol, e.g., gaboxadol monohydrate or gaboxadol hydrochloride is stored, e.g., in a reservoir (reservoir type) or dissolved in a liquid or gel-based reservoir (matrix type).

In embodiments, transdermal formulations may include chemical penetration enhancers and emulsions to facilitate transport of gaboxadol across the statum corneum. Examples of suitable penetration enhancers are alcohols, sulphoxides, azone, pyrrolidones, essential oils, terpenes and terpenoids, fatty acids, water and urea. In embodiments, semisolid vehicles such as proniosomes and microemulsion gels may be utilized as penetration enhancers. Proniosomes are non-ionic based surfactant vesicles, and may be known as “dry niosomes” since they can require hydration before drug release and permeation through the skin. Upon hydration proniosomes are converted into niosomes which are capable of diffusing across the stratum corneum and then adhere to the cell surface which causes a high thermodynamic activity gradient of the drug at the vesicle/stratum corneum surface, thus acting as the driving force for the penetration of drugs across the skin.

The starting point for the evaluation of the kinetics of drug release from a transdermal patch is an estimation of the drug compound's maximum flux across the skin (flux (J)) which is typically expressed in units of μg/cm²/h). Based on Fick's law of diffusion, the transport of gaboxadol molecules across skin will be maintained until the concentration gradient ceases to exist.

Accordingly, transdermal pharmaceutical formulations incorporating a reservoir will deliver a steady flux of gaboxadol across the membrane as long as excess undissolved drug remains in the reservoir. The time required for gaboxadol to reach a steady state of diffusion is called the lag time. In embodiments, matrix or monolithic devices may be characterized by a falling drug flux with time, as the matrix layers closer to the skin are depleted of drug. In embodiments, reservoir patches can include a porous membrane covering the reservoir of medication which can control release, while heat melting thin layers of medication embedded in the polymer matrix (e.g., the adhesive layer), can control release of drug from matrix or monolithic devices.

In embodiments, transdermal patches can include a release liner which protects the patch during storage and is removed prior to use, drug or drug solution in direct contact with the release liner, an adhesive which serves to adhere the components of the patch together along with adhering the patch to the skin, one or more membranes which can separate other layers, control the release of the drug from the reservoir and multi-layer patches, etc., and backing which protects the patch from the outer environment.

In embodiments, transdermal patches may include, but are not limited to, single-layer drug-in-adhesive patches, wherein the adhesive layer contains gaboxadol and serves to adhere the various layers of the patch together, along with the entire patch system to the skin, but is also responsible for the releasing of the drug; multi-layer drug-in-adhesive, wherein which is similar to a single-layer drug-in-adhesive patch, but contains multiple layers, for example, a layer for immediate release of the drug and another layer for controlled release of drug from the reservoir; reservoir patches wherein the drug layer is a liquid compartment containing a drug solution or suspension separated by the adhesive layer; matrix patches, wherein a drug layer of a semisolid matrix containing a drug solution or suspension which is surrounded and partially overlaid by the adhesive layer; and vapor patches, wherein an adhesive layer not only serves to adhere the various layers together but also to release vapor. Methods for making transdermal patches are described, e.g., in U.S. Pat. Nos. 6,461,644, 6,676,961, 5,985,311, and 5,948,433.

For example, an exemplary patch can include an impermeable backing bonded about its periphery to a permeation enhancer release rate controlling element and spaced apart therefrom in its central portion to define a permeation enhancer reservoir. The permeation enhancer release rate controlling element is similarly bonded about its periphery to a porous support member and spaced apart therefrom in its central portion to define an aqueous drug reservoir containing gaboxadol, which is water soluble. A contact adhesive layer which is permeable to the gaboxadol and enhancer can be bonded to the surface of porous support and a strippable release liner, adapted to protect the adhesive prior to use and can be readily removed therefrom, may also be provided. To permit transport of drug and enhancer to the skin, the adhesive may be porous or hydrated to be permeable to the drug and enhancer. If impermeable to drug and enhancer, the adhesive can be located or otherwise adapted to impose no significant resistance to drug and permeation enhancer transport to the skin. In embodiments, a porous polyacrylate adhesive can be utilized in the contact adhesive layer. If a hydratable contact adhesive formulation is used, the adhesive can be equilibrated with at least about 10 weight percent water to permit transport of ionized drug. It should be recognized, however, that if a peripherally located adhesive is used, it need not be porous or permeable. Also, if desired, an adhesive overlay or some other means such as buckles, belts, or elastic bands could be used to maintain the transdermal delivery device on the skin in which case, if properly packaged, the adhesive layer and the strippable release liner could be omitted. Such a system might be desirable, for example, if the drug adversely affected the adhesive properties of the adhesive layer or if the drug were highly soluble in the adhesive.

In embodiments, the aqueous reservoir containing the gaboxadol dispersed therein can contain at least 50%, e.g., 55%, 60%, 65%, 70%, 75%, 80%, 85% or 90%, water. In embodiments, the gaboxadol is present at a level above saturation. In embodiments, the reservoir can be in the form of a gel which may also contain stabilizing agents, other excipients and additives. A buffering agent may also be present if required to maintain the drug reservoir at physiological pH.

The permeation enhancer release rate controlling membrane controls the rate of release of the permeation enhancer from the permeation enhancer reservoir to the skin. In embodiments, a porous substrate functions as a physical support for the gelled aqueous reservoir and it should be sufficiently porous so that it imposes little or no resistance to the transport of drug and permeation enhancer to the skin. In this regard, viscosity of the aqueous reservoir can be related to the porosity of the porous substrate, i.e., it should be sufficiently viscous so that the aqueous reservoir will not readily flow through the porous substrate. The amount of gelling or other thickening agent used is not critical but should be an amount required to produce a viscosity in the aqueous reservoir sufficient to prevent the reservoir from migrating or otherwise leaking or oozing through the porous substrate. The porous adhesive is likewise selected to provide little or no resistance to drug or enhancer release. A function of the porous substrate is to provide a support to which the adhesive can be applied since it is difficult in many cases to provide a good bond between the porous adhesive and the aqueous medium within the reservoir. In embodiments, the rate controlling membrane can be a hydrophobic membrane which is capable of controlling the rate of release of the permeation enhancer from the enhancer reservoir while simultaneously preventing either water or the drug from diffusing or otherwise migrating into enhancer reservoir. In embodiments, upon standing, the aqueous drug reservoir can contain a saturation level of the permeation enhancer.

The impermeable backing can be any material which has the desired flexibility, impermeability and insolubility with respect to the permeation enhancer and may, e.g., either be a single element or a metalized or composite coated element. Suitable materials can include, without limitation, ethylene vinyl acetate copolymers (EVA), polyesters, metalized polyesters, polyethylenes, polycarbonates, polyvinyl chlorides, polyvinylidene fluoride, polysulfones, or laminates of the above such as metalized polyester/EVA or medium density polyethylene/EVA.

In embodiments, the porous substrate can be a soft, open-mesh, hydrophobic, fibrous material or may also be a non-fibrous, porous or sponge-like material as long as the substrate performs the function of being bondable to the adhesive and maintaining the gelled aqueous material within the reservoir without providing any significant resistance to the transport of drug and permeation enhancer. Examples of suitable materials include spun laced polyester, spun-laced polyolefin coated polyester, spun bonded polyethylene, spun laced polyethylene or EVA, microporous polypropylene, microporous polycarbonate, woven nylon, rayon or polyester cloths, and open cellular polyethylene or polyurethane foams.

The porous adhesive can be, e.g., a polyacrylate contact adhesive or any other suitable porous adhesive. Alternatively, the adhesive can be a non-porous contact adhesive which is applied about the periphery leaving the center portion beneath the aqueous reservoir substantially free of adhesive. In that case, any biocompatible contact adhesive could be applied, porous or not. Examples of adhesive compositions include silicone adhesives, polyacrylates, polyisobutylene-mineral oil adhesives, tackified styrene-isoprene-styrene block copolymers (SIS), tackified EVA contact adhesives, polyacrylamides and various hydratable, hot melt or emulsified (water borne) adhesive compositions.

The strippable release liner can be any material known to the art and may be the same as or different from the material used to provide the impermeable backing. A basic requirement for the strippable release liner is that it be substantially impermeable to the passage of components from the reservoir and be readily removed from the adhesive without destruction of the integrity of the patch.

With respect to the gelled aqueous drug reservoir, in the case of gaboxadol it is intended that water be the continuous phase. For that reason, the reservoir should be at least 50%, e.g., over 70% water. The gelling agent used to thicken the reservoir can be any of a wide variety of gelling agents, such as silica, particulate porous polyisoprene, bentonite clay, various gums such as agar, tragacanth, polysaccharides, cellulosic materials such as hydroxyethyl cellulose, hydroxypropyl cellulose or hydroxypropyl methyl cellulose and polyacrylates. The basic requirements are that the gelling agent is non-reactive with gaboxadol and does not substantially interfere with the ready diffusion of the materials from the patch. A relatively wide degree of flexibility in the amount of gelling agent used is available since the required viscosity varies inversely with the pore size selected for the substrate. A general range of approximately 1% to 10% by weight of these gelling agents may be adequate.

The drug reservoir may also contain a buffer to maintain the pH of the solution in a desired range during the drug delivery period. Suitable buffers should, of course, be unreactive with the other components of the system. Suitable buffers for acid drugs and basic drugs include, without limitation, phosphates, citrates, ascorbates and carbonates.

The permeation enhancer release rate controlling membrane should be substantially impermeable to the flow of water and gaboxadol from the aqueous reservoir into the permeation enhancer reservoir while having a sufficient degree of permeability to the permeation enhancer to permit the rate at which the permeation enhancer is released from the permeation enhancer reservoir into the skin to be controlled by membranes of reasonable thickness, e.g., in the range of 0.001-0.003 inches. The permeation enhancer release rate controlling membrane may either be a solid membrane or a microporous membrane having rate controlling material in the micropores to meter the release of permeation enhancer. Examples of rate controlling materials for the formation of a membrane per se or for the rate controlling material to be included in the pores of a microporous membrane can be, e.g., hydrophobic materials such as polyethylene EVA, polycarbonates, polyvinyl chloride, polyacrylate polymers, polysulfone polymers, polyvinylidienes, polyvinylidenes, polyesters, and polyisobutylenes.

The permeation enhancer may be present in the permeation enhancer reservoir either neat or as solution or dispersion in an appropriate medium. Exemplary materials include surfactants, such as alkyl substituted sulfoxides, e.g., n-octyl methyl sulfoxide, n-nonyl methyl sulfoxide, n-decylmethyl sulfoxide (n-DMS), n-undecyl methyl sulfoxide, n-dodecyl methyl sulfoxide; mono- and di-substituted alkyl polyethylene glycols such as polyethylene glycol mono laurate and polyethylene glycol di laurate; ethanol and other lower alcohols; n-methyl pyrrolidone, dimethyl lauramine, diethyltoluamide, and the 1-substituted azacycloalkan-2-ones.

In embodiments, active methods are utilized to drive penetration of gaboxadol through the stratum corneum. In embodiments, active methods for skin permeabilisation involve the use of external energy to act as a driving force for drug transport across the skin or by physically disrupting the stratum corneum. Active methods for skin permeabilisation include ultrasound, electrically assisted methods (electroporation and iontophoresis), velocity based devices (powder injection, jet injectors), thermal approaches (lasers and radio-frequency heating) and mechanical methodologies such as microneedles and tape stripping.

Embodiments described herein provide that a patient with essential tremor, Tourette syndrome or Fragile X syndrome and in need thereof is administered a modified release pharmaceutical formulation or a transdermal pharmaceutical formulation form including gaboxadol or a pharmaceutically acceptable salt thereof. Gaboxadol or pharmaceutically acceptable salt thereof may be provided as an acid addition salt, a zwitter ion hydrate, zwitter ion anhydrate, hydrochloride or hydrobromide salt, or in the form of the zwitter ion monohydrate. Acid addition salts, include but are not limited to, maleic, fumaric, benzoic, ascorbic, succinic, oxalic, bis-methylenesalicylic, methanesulfonic, ethane-disulfonic, acetic, propionic, tartaric, salicylic, citric, gluconic, lactic, malic, mandelic, cinnamic, citraconic, aspartic, stearic, palmitic, itaconic, glycolic, p-amino-benzoic, glutamic, benzene sulfonic or theophylline acetic acid addition salts, as well as the 8-halotheophyllines, for example 8-bromo-theophylline. In other suitable embodiments, inorganic acid addition salts, including but not limited to, hydrochloric, hydrobromic, sulfuric, sulfamic, phosphoric or nitric acid addition salts may be used.

In embodiments, gaboxadol is provided as gaboxadol monohydrate. One skilled in the art will readily understand that the amounts of active ingredient in a pharmaceutical formulation will depend on the form of gaboxadol provided. For example, pharmaceutical formulations including 5.0, 10.0, or 15.0 mg gaboxadol correspond to 5.6, 11.3, or 16.9 mg gaboxadol monohydrate.

In embodiments, gaboxadol is crystalline, such as the crystalline hydrochloric acid salt, the crystalline hydrobromic acid salt, or the crystalline zwitter ion monohydrate. In embodiments, gaboxadol is provided as a crystalline monohydrate.

Deuteration of pharmaceuticals to improve pharmacokinetics (PK), pharmacodynamics (PD), and toxicity profiles, has been demonstrated previously with some classes of drugs. Accordingly the use of deuterium enriched gaboxadol is contemplated and within the scope of the methods and formulations described herein. Deuterium can be incorporated in any position in replace of hydrogen synthetically, according to the synthetic procedures known in the art. For example, deuterium may be incorporated to various positions having an exchangeable proton, such as the amine N—H, via proton-deuterium equilibrium exchange. Thus, deuterium may be incorporated selectively or non-selectively through methods known in the art to provide deuterium enriched gaboxadol. See Journal of Labeled Compounds and Radiopharmaceuticals 19(5) 689-702 (1982).

Deuterium enriched gaboxadol may be described by the percentage of incorporation of deuterium at a given position in the molecule in the place of hydrogen. For example, deuterium enrichment of 1% at a given position means that 1% of molecules in a given sample contain deuterium at that specified position. The deuterium enrichment can be determined using conventional analytical methods, such as mass spectrometry and nuclear magnetic resonance spectroscopy. In some embodiments deuterium enriched gaboxadol means that the specified position is enriched with deuterium above the naturally occurring distribution (i.e., above about.0156%). In embodiments deuterium enrichment is no less than about 1%, no less than about 5%, no less than about 10%, no less than about 20%, no less than about 50%, no less than about 70%, no less than about 80%, no less than about 90%, or no less than about 98% of deuterium at a specified position.

In embodiments methods of treating a patient with essential tremor, Tourette syndrome or Fragile X syndrome include administering to a patient in need thereof a modified release pharmaceutical formulation or a transdermal pharmaceutical formulation including about 0.05 mg to about 100 mg gaboxadol or a pharmaceutically acceptable salt thereof.

In embodiments, the modified release pharmaceutical formulations or transdermal pharmaceutical formulations include 0.1 mg to 75 mg, 0.1 mg to 70 mg, 0.1 mg to 65 mg, 0.1 mg to 55 mg, 0.1 mg to 50 mg, 0.1 mg to 45 mg, 0.1 mg to 40 mg, 0.1 mg to 35 mg, 0.1 mg to 30 mg, 0.1 mg to 25 mg, 0.1 mg to 20 mg, 0.1 mg to 15 mg, 0.1 mg to 10 mg, 0.5 mg to 75 mg, 0.5 mg to 70 mg, 0.5 mg to 65 mg, 0.5 mg to 55 mg, 0.5 mg to 50 mg, 0.5 mg to 45 mg, 0.5 mg to 40 mg, 0.5 mg to 35 mg, 0.5 mg to 30 mg, 0.5 mg to 25 mg, 0.5 mg to 20 mg, 0.5 to 15 mg, 0.5 to 10 mg, 1 mg to 75 mg, 1 mg to 70 mg, 1 mg to 65 mg, 1 mg to 55 mg, 1 mg to 50 mg, 1 mg to 45 mg, 1 mg to 40 mg, 1 mg to 35 mg, 1 mg to 30 mg, 1 mg to 25 mg, 1 mg to 20 mg, 1 mg to 15 mg, 1 mg to 10 mg, 1.5 mg to 75 mg, 1.5 mg to 70 mg, 1.5 mg to 65 mg, 1.5 mg to 55 mg, 1.5 mg to 50 mg, 1.5 mg to 45 mg, 1.5 mg to 40 mg, 1.5 mg to 35 mg, 1.5 mg to 30 mg, 1.5 mg to 25 mg, 1.5 mg to 20 mg, 1.5 mg to 15 mg, 1.5 mg to 10 mg, 2 mg to 75 mg, 2 mg to 70 mg, 2 mg to 65 mg, 2 mg to 55 mg, 2 mg to 50 mg, 2 mg to 45 mg, 2 mg to 40 mg, 2 mg to 35 mg, 2 mg to 30 mg, 2 mg to 25 mg, 2 mg to 20 mg, 2 mg to 15 mg, 2 mg to 10 mg, 2.5 mg to 75 mg, 2.5 mg to 70 mg, 2.5 mg to 65 mg, 2.5 mg to 55 mg, 2.5 mg to 50 mg, 2.5 mg to 45 mg, 2.5 mg to 40 mg, 2.5 mg to 35 mg, 2.5 mg to 30 mg, 2.5 mg to 25 mg, 2.5 mg to 20 mg, 2.5 mg to 15 mg, 2.5 mg to 10 mg, 3 mg to 75 mg, 3 mg to 70 mg, 3 mg to 65 mg, 3 mg to 55 mg, 3 mg to 50 mg, 3 mg to 45 mg, 3 mg to 40 mg, 3 mg to 35 mg, 3 mg to 30 mg, 3 mg to 25 mg, 3 mg to 20 mg, 3 mg to 15 mg, 3 mg to 10 mg, 3.5 mg to 75 mg, 3.5 mg to 70 mg, 3.5 mg to 65 mg, 3.5 mg to 55 mg, 3.5 mg to 50 mg, 3.5 mg to 45 mg, 3.5 mg to 40 mg, 3.5 mg to 35 mg, 3.5 mg to 30 mg, 3.5 mg to 25 mg, 3.5 mg to 20 mg, 3.5 mg to 15 mg, 3.5 mg to 10 mg, 4 mg to 75 mg, 4 mg to 70 mg, 4 mg to 65 mg, 4 mg to 55 mg, 4 mg to 50 mg, 4 mg to 45 mg, 4 mg to 40 mg, 4 mg to 35 mg, 4 mg to 30 mg, 4 mg to 25 mg, 4 mg to 20 mg, 4 mg to 15 mg, 4 mg to 10 mg, 4.5 mg to 75 mg, 4.5 mg to 70 mg, 4.5 mg to 65 mg, 4.5 mg to 55 mg, 4.5 mg to 50 mg, 4.5 mg to 45 mg, 4.5 mg to 40 mg, 4.5 mg to 35 mg, 4.5 mg to 30 mg, 4.5 mg to 25 mg, 4.5 mg to 20 mg, 4.5 mg to 15 mg, 4.5 mg to 10 mg, 5 mg to 75 mg, 5 mg to 70 mg, 5 mg to 65 mg, 5 mg to 55 mg, 5 mg to 50 mg, 5 mg to 45 mg, 5 mg to 40 mg, 5 mg to 35 mg, 5 mg to 30 mg, 5 mg to 25 mg, 5 mg to 20 mg, 5 mg to 15 mg, or 5 mg to 10 mg, gaboxadol or a pharmaceutically acceptable salt thereof.

In embodiments, pharmaceutical formulations include 5 mg to 20 mg, 5 mg to 10 mg, 4 mg to 6 mg, 6 mg to 8 mg, 8 mg to 10 mg, 10 mg to 12 mg, 12 mg to 14 mg, 14 mg to 16 mg, 16 mg to 18 mg, or 18 mg to 20 mg gaboxadol or a pharmaceutically acceptable salt thereof.

In embodiments, modified pharmaceutical formulations or transdermal pharmaceutical formulations include 0.1 mg, 0.25 mg, 0.5 mg, 1 mg, 2.5 mg, 3 mg, 4 mg, 5 mg, 7 mg, 7.5 mg, 10 mg, 12.5 mg, 15 mg, 17.5 mg, or 20 mg gaboxadol or a pharmaceutically acceptable salt thereof or amounts that are multiples of such doses. In embodiments, modified pharmaceutical formulations or transdermal pharmaceutical formulations include 2.5 mg, 5 mg, 7.5 mg, 10 mg, 15 mg, or 20 mg gaboxadol or a pharmaceutically acceptable salt thereof.

In embodiments, ODDFs include 0.05 mg, 0.1 mg, 0.25 mg, 0.5 mg, 0.75 mg, 1 mg, 1.25 mg, 1.5 mg, 1.75 mg, 2 mg, 2.5 mg, 3 mg, 3.5 mg, 4 mg, 4.5 mg, 5 mg, 7 mg, 7.5 mg, 10 mg, 12.5 mg, 15 mg, 17.5 mg, or 20 mg gaboxadol or a pharmaceutically acceptable salt thereof or amounts that are multiples of such doses.

In embodiments, ERDFs include from about 1 mg to about 100 mg gaboxadol or a pharmaceutically acceptable salt thereof. In embodiments, ERDFs include 5 mg, 6 mg, 7 mg, 8 mg, 9 mg, 10 mg, 11 mg, 12 mg, 13 mg, 14 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, or 100mg gaboxadol or a pharmaceutically acceptable salt thereof.

In embodiments, delayed release dosage forms include from about 0.05 mg to about 100 mg gaboxadol or a pharmaceutically acceptable salt thereof. In embodiments, delayed release dosage forms include 0.05 mg, 0.1 mg, 0.25 mg, 0.5 mg, 0.75 mg, 1 mg, 1.25 mg, 1.5 mg, 1.75 mg, 2 mg, 2.5 mg, 3 mg, 3.5 mg, 4 mg, 4.5 mg, 5 mg, 6 mg, 7 mg, 8 mg, 9 mg, 10 mg, 11 mg, 12 mg, 13 mg, 14 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, or 100mg gaboxadol or a pharmaceutically acceptable salt thereof.

In embodiments, PRDFs include one or more pulse providing domains having from about 0.05 mg to about 100 mg gaboxadol or a pharmaceutically acceptable salt thereof. In embodiments, PRDFs include 0.05 mg, 0.1 mg, 0.25 mg, 0.5 mg, 0.75 mg, 1 mg, 1.25 mg, 1.5 mg, 1.75 mg, 2 mg, 2.5 mg, 3 mg, 3.5 mg, 4 mg, 4.5 mg, 5 mg, 6 mg, 7 mg, 8 mg, 9 mg, 10 mg, 11 mg, 12 mg, 13 mg, 14 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, or 100mg gaboxadol or a pharmaceutically acceptable salt thereof.

In embodiments, transdermal pharmaceutical formulations include from about 1 mg to about 100 mg gaboxadol or a pharmaceutically acceptable salt thereof. In embodiments, transdermal pharmaceutical formulations may include 5 mg, 6 mg, 7 mg, 8 mg, 9 mg, 10 mg, 11 mg, 12 mg, 13 mg, 14 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, or 100mg gaboxadol or a pharmaceutically acceptable salt thereof.

In embodiments, a modified release pharmaceutical formulation provides an in vivo plasma profile having Cₘₐₓ less than about 2500 ng/ml, 2000 ng/ml, 1750 ng/ml, 1500 ng/ml, 1250 ng/ml, 1000 ng/ml, 750 ng/ml, 500 ng/ml, 450 ng/ml, 400 ng/ml, 350 ng/ml, 300 ng/ml, 250 ng/ml, 200 ng/ml, 150 ng/ml, 100 ng/ml, 50 ng/ml or 25 ng/ml. In embodiments, ODDFs herein provide an in vivo plasma profile having a AUC₀₋₂₈ of less than about, e.g., 900 ng□hr/ml, 850 ng□hr/ml, 800 ng□hr/ml, 750 ng□hr/ml, or 700 ng□hr/ml 650 ng□hr/ml, 600 ng□hr/ml, 550 ng·hr/ml, 500 ng·hr/ml, or 450 ng·hr/ml . In embodiments, ODDFs herein provide an in vivo plasma profile having a A_{0-∞} of less than about, e.g., 400 ng□hr/ml, 350 ng·hr/ml, 300 ng□hr/ml, 250 ng□hr/ml, or 200 ng□hr/ml. In embodiments, ODDFs herein provide an in vivo plasma profile having a A_{0-∞} of less than about, e.g., 150 ng·hr/ml, 100 ng·hr/ml, 75 ng□hr/ml, or 50 ng□hr/ml. In embodiments, transdermal pharmaceutical formulations provide an in vivo plasma profile having Cₘₐₓ less than about 2500 ng/ml, 2000 ng/ml, 1750 ng/ml, 1500 ng/ml, 1250 ng/ml, 1000 ng/ml, 750 ng/ml, 500 ng/ml, 450 ng/ml, 400 ng/ml, 350 ng/ml, 300 ng/ml, 250 ng/ml, 200 ng/ml, 150 ng/ml, 100 ng/ml, 50 ng/ml or 25 ng/ml.

In embodiments, modified release pharmaceutical formulations with different drug release profiles may be combined to create a two phase or three-phase release profile. For example, as mentioned above, pharmaceutical formulations may be provided with an immediate release and an extended release profile. In embodiments, modified release pharmaceutical formulations may be provided with an immediate release, extended release and delayed release profile. Pharmaceutical formulations may be prepared using a pharmaceutically acceptable “carrier” composed of excipients that are considered safe and effective. The “carrier” includes all components present in the pharmaceutical formulation other than the active ingredient or ingredients. The term “carrier” includes, but is not limited to, excipients such as diluents, binders, lubricants, disintegrants, fillers, and coating formulations.

In embodiments, pharmaceutical formulations described herein are administered once, twice, three times daily, four times daily, every other day, every two days, every 3 days, every 4 days, every 5 days, every 6 days or every 7 days. In embodiments, a pharmaceutical formulation described herein is provided to the patient in the evening or in the morning. In embodiments, a pharmaceutical formulation described herein is provided to the patient once in the evening and once in the morning. In embodiments, the total amount of gaboxadol or a pharmaceutically acceptable salt thereof administered to a subject in a 24-hour period is 1 mg to 100 mg. In embodiments, the total amount of gaboxadol or a pharmaceutically acceptable salt thereof administered to a subject in a 24-hour period is 1 mg to 50 mg. In embodiments, the total amount of gaboxadol or a pharmaceutically acceptable salt thereof administered to a subject in a 24-hour period is 1 mg to 25 mg. In embodiments, the total amount of gaboxadol or a pharmaceutically acceptable salt thereof administered to a subject in a 24-hour period is 1 mg to 20 mg. In embodiments, the total amount of gaboxadol or a pharmaceutically acceptable salt thereof administered to a subject in a 24-hour period is 5 mg, 10 mg, or 15 mg. In embodiments, the total amount of gaboxadol or a pharmaceutically acceptable salt thereof administered to a subject in a 24-hour period is 20 mg.

In embodiments, provided herein are methods of treating essential tremor, Tourette syndrome or Fragile X syndrome including administering to a patient in need thereof a modified release pharmaceutical formulation or a transdermal pharmaceutical formulation including gaboxadol or a pharmaceutically acceptable salt thereof wherein the formulation provides improvement in at least one symptom of essential tremor, Tourette syndrome or Fragile X syndrome. Symptoms of Fragile X syndrome may include, but are not limited to, tremors such as intention tremor, resting tremor, rigidity, ataxia, bradykinesia, gait, speech impairment, vocalization difficulties, cognition impairment, motor activity deficits, clinical seizure, hypotonia, hypertonia, feeding difficulty, drooling, mouthing behavior, sleep difficulties, hand flapping, easily provoked laughter, short attention span, reduced sensation, numbness or tingling, pain, muscle weakness in the lower limbs, inability to control the bladder or bowel, chronic pain syndromes, such as fibromyalgia and chronic migraine, hypothyroidism, hypertension, sleep apnea, vertigo, olfactory dysfunction, and hearing loss, short-term memory loss, loss of executive function, impulse control, self-monitoring, focusing attention appropriately, cognitive flexibility psychiatric symptoms such as anxiety, depression, moodiness, or irritability. In embodiments, provided herein are improvements in cognition. Cognition refers to the mental processes involved in gaining knowledge and comprehension, such as thinking, knowing, remembering, judging, and problem solving. These higher-level functions of the brain encompass language, imagination, perception, and the planning and execution of complex behaviors.

Symptoms of Tourette syndrome include common tics such as eye blinking and other vision irregularities, throat clearing, grunting, facial grimacing, shoulder shrugging, and head or shoulder jerking. self-harm such as punching oneself, and vocal tics including coprolalia (uttering swear words) or echolalia (repeating the words or phrases of others).

Symptoms of essential tremor include rhythmic, muscle movement involving to-and-fro movements (oscillations) of one or more parts of the body, e.g., hand tremor, head tremor, arm tremor, voice tremor, tongue tremor, leg tremor, and trunk tremor. Head tremor may be seen as a “yes-yes” or “no-no” motion. Essential tremor may be accompanied by mild gait disturbance.

In embodiments, provided herein are methods of treating essential tremor, Tourette syndrome or Fragile X syndrome including administering to a patient in need thereof a ODDF including gaboxadol or a pharmaceutically acceptable salt thereof wherein the formulation provides improvement of at least one symptom within a half hour of administration. In embodiments, provided herein are methods of treating essential tremor, Tourette syndrome or Fragile X syndrome including administering to a patient in need thereof a ODDF including gaboxadol or a pharmaceutically acceptable salt thereof wherein the formulation provides improvement of at least one symptom within 45 minutes of administration. In embodiments, provided herein are methods of treating essential tremor, Tourette syndrome or Fragile X syndrome including administering to a patient in need thereof a ODDF including gaboxadol or a pharmaceutically acceptable salt thereof wherein the formulation provides improvement of at least one symptom within an hour of administration. In embodiments, provided herein are methods of treating essential tremor, Tourette syndrome or Fragile X syndrome including administering to a patient in need thereof a pharmaceutical formulation including gaboxadol or a pharmaceutically acceptable salt thereof wherein the formulation provides improvement of at least one symptom for more than 4 hours after administration of the pharmaceutical formulation to the patient. In embodiments, provided herein is improvement of at least one symptom for more than 6 hours after administration of the pharmaceutical formulation to the patient. In embodiments, provided herein is improvement of at least one symptom for more than, e.g., 8 hours, 10 hours, 12 hours, 15 hours, 18 hours, 20 hours, or 24 hours after administration of the pharmaceutical formulation to the patient. In embodiments, provided herein is improvement in at least one symptom for at least, e.g., 8 hours, 10 hours, 12 hours, 15 hours, 18 hours, 20 hours, or 24 hours after administration of the pharmaceutical formulation to the patient. In embodiments, provided herein is improvement in at least one symptom for 12 hours after administration of the pharmaceutical formulation to the patient.

Unless defined otherwise, all technical and scientific terms used herein have the same meanings as commonly understood by one of skill in the art to which the disclosure herein belongs.

The term “about” or “approximately” as used herein means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, up to 10%, up to 5%, or up to 1% of a given value. Alternatively, particularly with respect to biological systems or processes, the term can mean within an order of magnitude, preferably within 5-fold, and more preferably within 2-fold, of a value.

“Improvement” refers to the treatment of a subject having essential tremor, Tourette syndrome or Fragile X syndrome measured relative to at least one symptom.

“PK” refers to the pharmacokinetic profile. Cₘₐₓ is defined as the highest plasma drug concentration estimated during an experiment (ng/ml) following administration of a drug. Tₘₐₓ is defined as the time when Cₘₐₓ is estimated (min). AUC_{0-∞} is the total area under the plasma drug concentration-time curve, from drug administration until the drug is eliminated (ng□hr/ml). The area under the curve is governed by clearance. Clearance is defined as the volume of blood or plasma that is totally cleared of its content of drug per unit time (ml/min).

“Treating” or “treatment” refers to alleviating or delaying the appearance of clinical symptoms of a disease or condition in a subject that may be afflicted with or predisposed to the disease or condition, but does not yet experience or display clinical or subclinical symptoms of the disease or condition. In certain embodiments, “treating” or “treatment” may refer to preventing the appearance of clinical symptoms of a disease or condition in a subject that may be afflicted with or predisposed to the disease or condition, but does not yet experience or display clinical or subclinical symptoms of the disease or condition. “Treating” or “treatment” also refers to inhibiting the disease or condition, e.g., arresting or reducing its development or at least one clinical or subclinical symptom thereof. “Treating” or “treatment” further refers to relieving the disease or condition, e.g., causing regression of the disease or condition or at least one of its clinical or subclinical symptoms. The benefit to a subject to be treated may be statistically significant, mathematically significant, or at least perceptible to the subject and/or the physician. Nonetheless, prophylactic (preventive) and therapeutic (curative) treatment are two separate embodiments of the disclosure herein.

“Pharmaceutically acceptable” refers to molecular entities, formulations and compositions that are “generally regarded as safe”, e.g., that are physiologically tolerable and do not typically produce an allergic or similar untoward reaction, such as gastric upset and the like, when administered to a human. In embodiments, this term refers to molecular entities, formulations and compositions approved by a regulatory agency of the federal or a state government, as the GRAS list under section 204(s) and 409 of the Federal Food, Drug and Cosmetic Act, that is subject to premarket review and approval by the FDA or similar lists, the U.S. Pharmacopeia or another generally recognized pharmacopeia for use in animals, and more particularly in humans.

“Effective amount” or “therapeutically effective amount” means a dosage sufficient to alleviate one or more symptom of a disorder, disease, or condition being treated, or to otherwise provide a desired pharmacological and/or physiologic effect.

“Pharmaceutical formulations” includes dosage forms and unit doses.

“Patient in need thereof” may include individuals that have been diagnosed with essential tremor, Tourette syndrome or Fragile X syndrome. The methods may be provided to any individual including, e.g., wherein the patient is a neonate, infant, a pediatric patient (6 months to 12 years), an adolescent patient (age 12-18 years) or an adult (over 18 years).

The following examples are included to help illustrate and/or augment the description herein. The examples are not to be construed as limiting the disclosure herein in any way.

### EXAMPLE 1

### Prospective Assessment of Efficacy of Treatment of Essential Tremors with Gaboxadol 15mg Orally Disintegrating Tablets 15 mg

| | | | Unit Strength |
|---|---|---|---|
| Components | Compendial | | 15 mg |
| Intragranular | Testing | Function | mg/tablet |
| Gaboxadol^{†}(equivalent | — | Active | 16.94 |
| anhydrous) | | | |
| Aspartame | NF | Sweetener | 2.00 |
| Peppermint (Natural | — | Flavor | 1.00 |
| and Artificial) | | | |
| Monoammonium | — | Sweetener | 1.00 |
| Glycyrrhizinate | | | |
| Lactose Monohydrate | NF | Diluent | 63.87 |
| (modified spray-dried) | | | |
| Crospovidone | NF | Disintegrant | 10.00 |
| Mannitol | USP | Diluent | 104.00 |
| FD&C Blue No. 2 | — | Colorant | 0.20 |
| Aluminum Lake | | | |
| Magnesium Stearate | NF | Lubricant | 1.00 |
| (non-bovine) | | | |
| Total Tablet Weight | | | 200.0 |

^{†}Conversion Factor: 1.129 mg of monohydrate = 1.0 mg of anhydrous

The gaboxadol ODT formulation is prepared by blending the active drug, aspartame, peppermint flavor, monoammonium glycyrrhizinate, lactose monohydrate, crospovidone, mannitol and FD&C blue #2 in a suitable diffusional blender until uniform. The magnesium stearate is added and the material is blended. The final lubricated blend is compressed on a tablet press.

15 mg gaboxadol ODTs prepared as above is to be utilized in a double-blind, placebo-controlled, parallel-group study. Subjects will be randomized to one of two treatment groups. Group A will receive 15 mg gaboxadol ODTs and Group B will receive placebo ODTs. Subject randomization will be stratified by concomitant primidone use and site type (sub-study vs non sub-study). Tremor will be assessed via The Essential Tremor Rating Assessment Scale (TETRAS) and accelerometry. In order to reduce rater bias, all subjects will be videotaped during the TETRAS performance scale testing according to a consistent script. The videotapes will be rated in a blinded manner. A subset of subjects will participate in an electroencephalography (EEG) and magnetoencephalography (MEG) sub-study to record power-spectral brain activity in specific neuro-anatomical locations and coherence with movement measures. Subjects will be screened up to one month prior to initiation of dosing. At Baseline, subjects will undergo safety and tremor assessments prior to dosing, will receive their first dose of study drug and will be monitored for safety for one hour following dosing. For one week subjects will receive one 15 mg ODT (or matching placebo) daily. Subjects will return to the clinic on Day 8 for safety monitoring. At Day 15 (Week 3) subjects will return to clinic for safety and efficacy assessments. The final efficacy visit will occur at Day 28 (Week 4). A final safety visit will occur at Day 35 (Week 5).

### EXAMPLE 2

### Prospective Assessment of Treatment of Essential Tremors With Gaboxadol Orally Disintegrating Film 10 mg

A hydrophilic film-forming agent is made from a graft copolymer having a film-forming block of polyvinyl alcohol (PVA) Kollicoat IR® (marketed by BASF), molecular weight about 45,000 Da, and a polyethylene glycol (PEG) plasticizer. The gelling agent is Gelcarin 379® (commercially available from FMC Biopolymer), a compound of the carrageenan family. Kollicoat IR® is introduced into 70% of the amount of purified water under stirring. Agitation is maintained until dissolution of Kollicoat IR®. Since gas bubbles are generated, the solution may be dissolved under a vacuum or the solution can stand (its viscosity is very low) until the gas is dispersed. Tween 80 is incorporated to the stirred solution and flavorings (condensed licorice extract and essential oil of peppermint) and sweetener (acesulfame potassium) are added. Stirring is continued until complete dissolution of all powder. Gaboxadol monohydrate 10 mg is introduced with stirring until it is dispersed in the mixture, then the remaining water (30%) is added. Gelcarin 379® is incorporated into suspension under agitation to prevent the formation of aggregates. The final mixture consists of gaboxadol 10 mg, Kollicoat IR® 15% w/w, Gelcarin 379® 5% w/w, Tween 80 0.2% w/w, acesulfame potassium 0.05% w/w, flavorings 1.5% w/w, purified water qs. Mixing aliquots are then coated on a polyester backing and dried in a type Lab Dryer Coater (Mathis equipment). The coated surfaces are cut using a manual press in 6 cm² units, and then manually packaged in sealed bags.

10 mg gaboxadol ODFs prepared as above will be utilized in a double-blind, placebo-controlled, parallel-group study. Subjects will be randomized to one of two treatment groups. Group A will receive 10 mg gaboxadol ODFs and Group B will receive placebo ODFs. Subject randomization will be stratified by concomitant primidone use and site type (sub-study vs non sub-study). Tremor will be assessed via The Essential Tremor Rating Assessment Scale (TETRAS) and accelerometry. In order to reduce rater bias, all subjects will be videotaped during the TETRAS performance scale testing according to a consistent script. The videotapes will be rated in a blinded manner. A subset of subjects will participate in an electroencephalography (EEG) and magnetoencephalography (MEG) sub-study to record power-spectral brain activity in specific neuro-anatomical locations and coherence with movement measures. Subjects will be screened up to one month prior to initiation of dosing. At Baseline, subjects will undergo safety and tremor assessments prior to dosing, will receive their first dose of study drug and will be monitored for safety for one hour following dosing. For one week subjects will receive one 10 mg ODF (or matching placebo) daily. Subjects will return to the clinic on Day 8 for safety monitoring. At Day 15 (Week 3) subjects will return to clinic for safety and efficacy assessments. The final efficacy visit will occur at Day 28 (Week 4). A final safety visit will occur at Day 35 (Week 5).

### EXAMPLES 3 and 4

### Transdermal Patch Fabrication

Transdermal delivery devices for the delivery of gaboxadol monohydrate and gaboxadol hydrochloride are fabricated as set forth below (percentages in weight %.). The systems can be fabricated in sizes of from 3 cm² to 40 cm². When applied to the chest of a patient, a projected steady state delivery rate in the ranges shown may be established after approximately 2-7 hours and maintained for the projected periods shown.

### EXAMPLE 3

Backing: Polyester/EVA laminate
Permeation enhancer: 50% n-DMS-50% EVA (40% VA)
Reservoir: Loading 40 mg/cm²
Rate Control Membrane: EVA (12% VA) 2 mil thick
Drug: 25% gaboxadol monohydrate
Reservoir: 3% hydroxypropyl cellulose (gellant)—67% water Loading 30 mg/cm²
Support membrane: Porous polypropylene—2 mil thick
Adhesive: In-line porous polyacrylate
Steady State In Vivo Release Rate: 15 μg/cm² hr for 1-3 days

### EXAMPLE 4

Backing: Medium Density Polyethylene Polyester/EVA
Trilaminate
Permeation enhancer: 50% n-DMS-50% EVA (40% VA)
Reservoir: Loading 40 mg/cm²
Rate Control Membrane: EVA (12% VA) 2 mil thick
Drug: 30% gaboxadol HCl
Reservoir: 5% hydroxypropylmethyl cellulose (gellant)—80% water
Loading 25 mg/cm²
Support membrane: Spun based EVA/polyester
Adhesive: In-line porous polyacrylate
Steady State In Vivo
Release Rate: 20 μg/cm² hr for 1-3 days

While embodiments of the disclosure have been described and exemplified herein, it is not intended that the disclosure be limited thereto, as it is intended that the disclosure be as broad in scope as the art will allow and that the specification be read likewise. Therefore, the above description should not be construed as limiting, but merely as exemplifications of various embodiments. Those skilled in the art will envision other modifications within the scope and spirit of the claims appended hereto.

What is claimed is:

1. A method of treating essential tremors comprising administering to a patient in need thereof a pharmaceutical formulation comprising about 0.05 mg to about 100 mg gaboxadol or a pharmaceutically acceptable salt thereof wherein the formulation provides a Tₘₐₓ of less than 20 minutes.

2. The method according to claim 1, wherein the pharmaceutical formulation is an orally disintegrating dosage form.

3. The method according to claim 2, wherein the orally disintegrating dosage form is an orally disintegrating tablet, an orally disintegrating film, an orally disintegrating wafer or an orally disintegrating capsule.

4. A method of treating essential tremors comprising administering to a patient in need thereof a modified release pharmaceutical formulation comprising about 0.05 mg to about 100 mg gaboxadol or a pharmaceutically acceptable salt thereof wherein the formulation provides sustained delivery of gaboxadol or a pharmaceutically acceptable salt thereof for more than 4 hours.

5. The method according to claim 4, wherein the formulation provides sustained delivery of gaboxadol or a pharmaceutically acceptable salt thereof for more than 8 hours.

6. A method of treating essential tremors comprising administering to a patient in need thereof a transdermal pharmaceutical formulation comprising about 0.05 mg to about 100 mg gaboxadol or a pharmaceutically acceptable salt thereof wherein the transdermal pharmaceutical formulation comprises a patch.

7. The method according to claim 6, wherein the patch delivers a sustained dose of gaboxadol or a pharmaceutically acceptable salt thereof over a period ranging from 1 day to one week.
